(19) United States
(12) Patent Application Publication
(10) Pub. No.: US 2021/0221774 A1
(43) Pub. Date: Jul. 22, 2021

(54) TREATMENT OF POST-TRAUMATIC SYNDROME DISORDER

(71) Applicant: Aché Laboratórios Farmacêuticos S.A, Guarulhos (BR)

(72) Inventors: Alessandra MASCARELLO, São Paulo (BR); Cristiano Ruch Werneck GUIMARÃES, São Paulo (BR); Hatylas Felype Zaneti de AZEVEDO, São Paulo (BR); Marcos Antonio Ferreira JUNIOR, São Paulo (BR)

(21) Appl. No.: 16/768,991

(22) PCT Filed: Dec. 14, 2018
(86) PCT No.: PCT/BR2018/050461

§ 371 Date: Jun. 2, 2020

Related U.S. Application Data
(60) Provisional application No. 62/598,757, filed on Dec. 14, 2017.

(51) Int. Cl. C07D 239/90; A61P 25/24; A61P 25/22; A61K 45/06

(52) U.S. Cl. CPC C07D 239/90; A61K 45/06; A61P 25/22; A61P 25/24

(57) ABSTRACT

This invention is related to 3-(2-(4-(2-methoxyphenyl)piperazine-1-yl) ethyl) quinazoline-4(3H)-one, pharmaceutically acceptable salts, enantiomers, diasteroisomers, stereoisomers, crystals, hydrates, solvates, prodrugs, and metabolites thereof for the treatment of Post-Traumatic Syndrome Disorder (PTSD), and pharmaceutical compositions containing the compounds for the treatment of Post-Traumatic Syndrome Disorder (PTSD).

### PRIOR ART

Treating mental health illnesses has always been a challenge, because of all the complexity involved, both from the psychiatric/psychological side and the medication side, and the difficult balance between them.

While the psychiatric/psychological treatment requires highly skilled professionals to detect the symptoms of each disease, as well as the co-existence of diseases (such as anxiety disorders, mood disorders, psychotic disorders, eating disorders, impulse control and addiction disorders, personality disorders, obsessive-compulsive disorder, post-traumatic stress disorder, stress response syndromes, dissociative disorders, factitious disorders, sexual and gender disorders, tic disorders, etc.), psychiatric medication does not cure mental illnesses, even if it can significantly improve symptoms.

Presently, regardless of what they are and how they manifest themselves, most of those disorders are treated with antidepressant and/or anti-stress medication.

This invention is specifically related to medication for post-traumatic stress disorder (PTSD), which is a chronic anxiety disorder that follows an exposure to traumatic events. Nearly everyone will experience a range of reactions after trauma, yet most people recover from initial symptoms naturally. Those who continue to experience problems may be diagnosed with PTSD. People who have PTSD may feel stressed or frightened even when they are not in danger.

Not every traumatized person develops ongoing (chronic) or even short-term (acute) PTSD. Not everyone with PTSD has been through a dangerous event. Some experiences, like the sudden, unexpected death of a loved one, can also cause PTSD. Symptoms usually begin early, within 3 months of the traumatic incident, but sometimes they begin years afterward. To be considered a case of PTSD, symptoms must last for more than one month and be severe enough to interfere with relationships or work. The course of the illness varies. Some people recover within 6 months, while others have symptoms that last much longer. In some people, the condition becomes chronic. Very typically, war veterans are affected by PTSD.

The most commonly used medications for PTSD are well known antidepressants, which relieve symptoms of depression and anxiety. Selective serotonin reuptake inhibitors (for example, fluoxetine) are typically the first line treatment, and are often prescribed for the treatment of PTSD. Tricyclic antidepressants (for example, desipramine) or monoamine oxidase inhibitors are generally resorted as second- and third-line strategies due to tolerability issues.

Other anti-depression drugs have also been recommended to treat PTSD, such as benzodiazepines. No data is known to support the efficacy of benzodiazepines for the treatment of what is considered “core” PTSD symptoms such as avoidance, hyperarousal, numbing and dissociation. Nevertheless, they are commonly prescribed, probably because they presumably manage secondary symptoms of PTSD such as insomnia and anxiety due to their rapid short-term symptomatic relief.

Unfortunately, many PTSD patients fail to adequately respond to the existing pharmacological treatments with only about 60% patients responding to treatment to some degree and approximately 20-30% who achieve full remission. Thus, it seems that the available pharmacotherapies do not offer a sufficient solution for PTSD patients and there is a major need for novel treatment strategies.

Additionally, because antidepressant medication constitutes the first line pharmacological treatment for PTSD and many patients display no beneficial drug effects, it has been suggested that combinations of antidepressants with additional drugs may be necessary. Indeed, the heterogeneity of symptom clusters in PTSD, as well as the complex psychiatric comorbidities (for example, with depression or substance abuse), further support the notion that combinations of medications are in need. Therefore, the present consensus for effective treatment of PTSD and its complex psychiatric comorbidities is a combination of treatments.

However, a combination of medications to treat complex behavior disorders such as PTSD is not without drawbacks, as one has to deal with two sets of drug side effects instead of one as well as drug-drug interactions.

Thus, it seems that the available pharmacotherapies do not offer a sufficient solution for PTSD patients and there is a major need for novel treatment strategies.

### DETAILS OF THE INVENTION

A research investigation found that a certain quinazolinone compound provides effective treatment for PTSD symptoms, more effectively than drugs presently employed in such a treatment, with fewer side effects.

The quinazolinone compound of the invention is 3-(2-(4-(2-methoxyphenyl)piperazine-1-yl) ethyl) quinazoline-4(3H)-one, with the structure below:

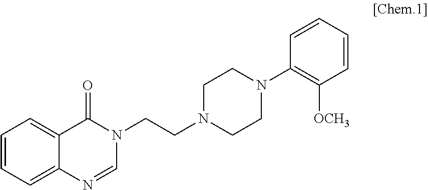 [SMILES: COc1ccccc1N1CCN(CCn2cnc3ccccc3c2=O)CC1]

Within the meaning of the quinazolinone compound of the invention are also included pharmaceutically acceptable salts, enantiomers, diasteroisomers, stereoisomers, crystals, hydrates and solvates thereof, provided they significantly afford the activity of the compound concerning PTSD treatment. Within the meaning of the quinazolinone compound of the invention are also included prodrugs and metabolites thereof, as well as the free base molecule with minor modifications (e.g. inclusion of substituents), which, as known to a person having skill in the art, do not significantly modify the pharmacophore of the molecule towards PTSD.

Without excluding any other alternatives, adequate salts of the quinazolinone compound of the invention are pharmaceutically acceptable acid addition salts thereof, such as inorganic addition salts e.g. hydrochloride, hydrobromide, sulfate, nitrate, phosphate, and pharmaceutically acceptable organic additions salts such as acetate, propionate, hexanoate, heptanoate, glycolate, piruvate, lactate, malonate, malate, maleate, fumarate, tartrate, citrate, succinate, mesylate, acistrate, besylate, tosylate, xinafoate, benzoate, p-toluenesulfonate, cinnamate, p-chlorobenzenesulfonate, 2-naphtalenesulfonate, p-toluenesulphonate, camphorsulfonate, trimethylacetate, t-butylacetate, laurylsulphate, gluconate, glutarate, hydroxynaphthoate, salicylate, stearate, muconate, mandelate, 2-hydroxyethanesulfonate.

Without excluding any other alternatives, adequate salts of the quinazolinone compound of the invention are pharmaceutically acceptable alkaline addition salts thereof, for instance alkaline metal salts such as sodium, potassium, lithium, calcium, magnesium, bismuth, bromide, or pharmaceutically acceptable salts with organic bases such as primary, secondary or tertiary amines (e.g. isopropylamine, diethylamine, trimethylamine, triisopropylamine, tri-n-propylamine, ethanolamine, 2-dimethylaminoethanol, tromethamine, N-methyl-D-glucamine) and with amino acid salts such as arginine, lysine, histidine, caffeine, procaine, hydro amine, choline, betaine, ethylene diamine, glucosamine, theobromine, purine, morphine.

The quinazolinone compound or the invention can be prepared by known methods. For instance it can be prepared as illustrated in diagram 1 below:

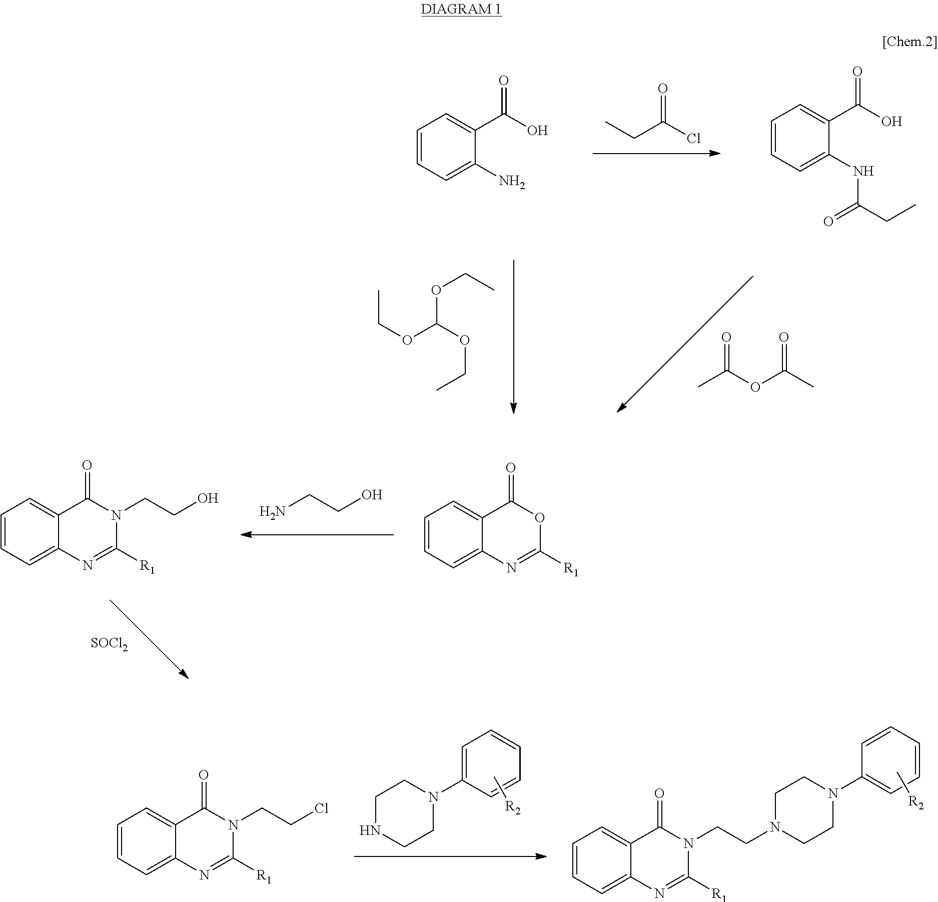

Wherein R1 is hydrogen and R2 is methoxyphenyl.

Pursuant to diagram 1, the compound of the invention may be obtained from anthranilic acid, using ethyl orthoformate and monoethanolamine for the formation of the intermediate 3-(2-hydroxyethyl)quinazoline-4 (3H)-one. The reaction of this intermediate with thionyl chloride results in the formation of a chlorinated derivative, which is subsequently condensed with a substituted piperazine phenyl resulting in the desired compound. Other synthetic alternatives may be used to obtain it, without adversely affecting the outcome.

One aspect of the invention is the quinazolinone compound as defined above.

Another aspect of the invention is the treatment of PTSD characterized by the administration of a therapeutically efficient amount of the quinazolinone compound to a person in need thereof.

Another aspect of the invention is a PTSD-treating pharmaceutical composition comprising the quinazolinone compound as defined above, and one or more excipients.

The selection of excipients to be used or preparing pharmaceutical compositions is generally undertaken by considering the type of administration pathway, the physical and chemical compatibility of the excipient with the active ingredient, the manner of preparing the pharmaceutical presentation and the effects on its efficacy. These excipients are known in the art and are described in the literature (for instance in: Handbook of Pharmaceutical Manufacturing Formulations—Vol. 1 a6-2004 Sarfaraz K. Niazi—CRC Press and Remington's Pharmaceutical Sciences, Mack Publishing), widely used by technical professionals in the matter.

For therapeutic use and administration, the quinazolinone compound of the invention may be formulated in compositions appropriate for oral, parenteral, nasal, rectal, transmucosal and transdermal administration, using conventional techniques and appropriate excipients.

There are no specific restrictions as to the dosage forms comprising the quinazolinone compound of the invention. For instance, as a solid for oral administration adequate dosage forms may be tablets, pills, dragées, capsules, granules, powders, pellets, lyophilizates and similar presentations. As a liquid for oral administration, adequate dosage forms may be solutions, dispersions, suspensions, emulsions, oils, syrups, etc. Liquid dosage forms may be used for injections, such as intravenous, intramuscular, subcutaneous and intradermal.

Other examples of dosage forms comprise liposomes and nanoparticles, or any other known administration aspects to a person skilled in the art. The dosage form may provide immediate, controlled or delayed release of the quinazolinone compound of the invention.

Another aspect of the invention are dosage forms, as described above, containing 0.01 a 5000 mg of the quinazolinone compound of the invention, for example, and may be administered once or more times a day, during the treatment.

In a particular embodiment, the composition of the invention may comprise, other than the quinazolinone compound, at least another active principle, for instance chosen from anti-depressant, anxiolytic, anti-retroviral, anti-cancer, anti-AIDS, antibiotic, antidiabetic, anti-hypertensive, analgesic, anti-inflammatory.

In another aspect, the invention concerns the use of the quinazolinone compound described above in the preparation of a medicament to treat PTSD.

In another aspect, the invention concerns the quinazolinone compound described above for the treatment of PTSD.

### BRIEF DESCRIPTION OF DRAWINGS

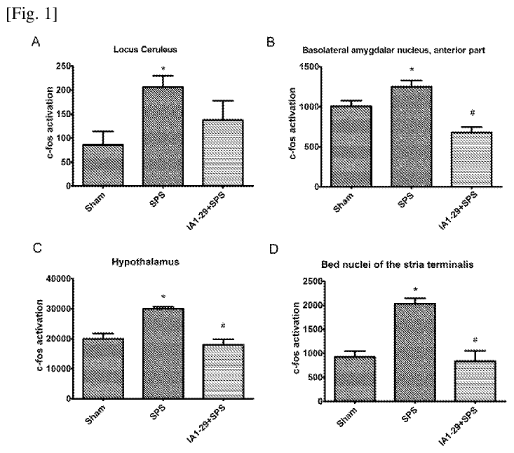

Count of c-fos positive neurons in brain regions. The brain regions exemplified in FIG. 1 are locus ceruleus (FIG. 1A), amygdala (FIG. 1B), hypothalamus (FIG. 1C) and bed nuclei stria terminalis (FIG. 1D). The experiment comprised three different groups (n=10 per group): mice not subjected to the Single-Prolonged Stress protocol (Sham), mice subjected to the Single-Prolonged Stress protocol (SPS) and mice subjected to the Single-Prolonged Stress protocol but pre-treated with 3-(2-(4-(2-methoxyphenyl)piperazine-1-yl) ethyl) quinazoline-4(3H)-one, named IA1-29, at 30 mg/kg (IA1-29+SPS).

### EXAMPLE

The quinazolinone of the invention was named IA1-29 and was tested in an in vivo model that mimics the activation that occurs in the brain after a prolonged stress stimulus.

The effects of the administration of IA1-29 at 30 mg/kg p.o. in brain activation were assessed in mice subjected to the Single Prolonged Stress (SPS) model. The SPS protocol is a well established model of PTSD that combines multiple stressors (physical restraint, forced swim, predator scent and ether anesthesia (Borghans & Homberg, 2015; Yamamoto et al., 2009)). The activation of specific brain regions was determined by counting the number of neurons expressing the c-fos protein, an immediate early gene product that can be used as a marker of cell activation in individual neurons. Interestingly, the SPS-Induced c-fos expression in key brain regions involved in stress response was suppressed by the prior administration of IA1-29. The count of c-fos positive neurons in some of these regions can be visualized in FIG. 1. The experiment comprised three different groups (n=10 per group): mice not subjected to the SPS protocol (Sham), mice subjected to the SPS model (SPS) and animals submitted to the SPS protocol but pre-treated with IA1-29 at 30 mg/kg (IA1-29+SPS). The brain regions exemplified in FIG. 1 are locus ceruleus (FIG. 1A), amygdala (FIG. 1B), hypothalamus (FIG. 1C) and bed nuclei stria terminalis (FIG. 1D). The statistical analysis between the groups was performed by ANOVA followed by bonferroni post-hoc test. * p<0.05 between SPS and Sham groups. # p<0.05 between IA1-29+SPS and SPS groups.

A person having skills in the concerned art, by way of the explanations and examples comprised herein will promptly appreciate the advantages of the invention, and will be able to propose equivalent embodiments of the invention without departing from the scope of the attached inventions.

1. A quinazoline compound having the formula 3-(2-(4-(2-methoxyphenyl)piperazine-1-yl) ethyl) quinazoline-4(3H).

2. A pharmaceutical composition for treating Post-Traumatic Syndrome Disorder (PTSD) comprising the quinazoline compound according to claim 1 and pharmaceutically acceptable salts, enantiomers, diasteroisomers, stereoisomers, crystals, hydrates, solvates, prodrugs, or metabolites thereof.

3. The pharmaceutical composition according to claim 2 that does not contain any ingredients that would modify the phannacophore for the treatment of Post-Traumatic Syndrome Disorder.

4. The pharmaceutical composition according to claim 2 wherein the pharmaceutically acceptable salts are selected from the group consisting of inorganic addition salts selected from the group consisting of hydrochloride, hydrobromide, sulfate, nitrate, and phosphate; organic addition salts selected from the group consisting of acetate, propionate, hexanoate, heptanoate, glycolate, piruvate, lactate, malonate, malate, maleate, lumarate, tartrate, citrate, succinate, mesylate, acistrate, besylate, tosylate, xinafoatc, benzoate, p-toluenesulfonate, cinnamate, p-chlorobenzenesulfonate, 2-naplitalenesulfonate, p-toluenesulphonate, camphorsulfonate, trimethylacetate, t-butylacetate, laurylsulphate, gluconate, glutarate, hydroxynaphthoate, salicylate, stearate, muconate, mandelate, and 2-hydroxyethanesulfonate; alkaline addition salts of metals selected from the group consisting of sodium, potassium, lithium, calcium, magnesium, bismuth, and bromide; salts with the organic bases primary, secondary or tertiary amines; and salts with an amino acid selected from the group consisting of arginine, lysine, histidine, caffeine, procaine, hydrobamine, choline, betaine, ethylenediamine, glycosamine, teobromine, purine, morpholine.

5. A method of treating Post-Traumatic Syndrome Disorder (PTSD) comprising administering a therapeutically effective amount of the quinazolinone compound according to claim 1 to a person in need thereof.

6. A pharmaceutical composition for treating Post-Traumatic Syndrome Disorder (PTSD) comprising the quinazolinone compound according to claim 1 and one or more excipients.

7. The pharmaceutical composition according to claim 6 further comprising at least one additional active principle, selected from the group consisting of anti-depressant, anxiolytic, anti-retroviral, anti-cancer, antibiotic, antidiabetic, anti-hypertensive, analgesic, and anti-inflammatory active principles.

8. A dosage form comprising the quinazolinone compound according to claim 1 in an amount of 0.01 to 5000 mg.

9. A method of using the quinazolinone compound according to claim 1 comprising using said compound in the treatment of Post-Traumatic Syndrome Disorder (PTSD).

10. A method of treating Post-Traumatic Syndrome Disorder (PTSD) comprising administering a therapeutically effective amount of the pharmaceutical composition according to claim 2 to a person in need thereof.

11. A method of treating Post-Traumatic Syndrome Disorder (PTSD) comprising administering a therapeutically effective amount of the pharmaceutical composition according to claim 6 to a person in need thereof.

12. A dosage form comprising the pharmaceutical compound according to claim 2 wherein the quinazoline compound is present in an amount of 0.01 to 5000 mg.

13. A dosage form comprising the pharmaceutical compound according to claim 3 wherein the quinazoline compound is present in an amount of 0.01 to 5000 mg.

14. A dosage form comprising the pharmaceutical compound according to claim 4 wherein the quinazoline compound is present in an amount of 0.01 to 5000 mg.

15. A pharmaceutical composition according to claim 2 further comprising at least one additional active principle selected from the group consisting of anti-depressant, anxiolytic, anti-retroviral, anti-cancer, antibiotic, antidiabetic, anti-hypertensive, analgesic, and anti-inflammatory active principles.
